(19) United States
(12) Patent Application Publication
(10) Pub. No.: US 2019/0231269 A1
(43) Pub. Date: Aug. 1, 2019

(54) SENSORIZED GUIDEWIRE AND CATHETER

(71) Applicants: Caroline CAO, Dayton, OH (US); WRIGHT STATE UNIVERSITY, Dayton, OH (US)

(72) Inventor: Caroline CAO, Dayton, OH (US)

(21) Appl. No.: 16/332,248

(22) PCT Filed: Oct. 5, 2017
(86) PCT No.: PCT/US17/55346

§ 371 Date: Mar. 11, 2019

Related U.S. Application Data
(60) Provisional application No. 62/404,370, filed on Oct. 5, 2016.

(51) Int. Cl. A61B 5/00; G01L 1/24; A61M 25/09; A61B 5/06

(52) U.S. Cl. CPC A61B 5/6851; G01L 1/242; A61M 25/09; G02B 6/3624; A61B 5/6862; A61B 5/0093; A61B 5/065

(57) ABSTRACT

A sensorized surgical tool including a fiber optic shape tracking sensor for endovascular applications and methods of making the same.

### CROSS-REFERENCE TO RELATED APPLICATIONS

This application claims the benefit of U.S. Provisional Patent Application Ser. No. 62/404,370 entitled “SENSORIZED GUIDEWIRE AND CATHETER” filed on Oct. 5, 2016, the entirety of which is incorporated by reference herein.

### BACKGROUND

Guidewires and/or catheters are an integral part of vascular intervention. They are utilized to access target vessels, cross lesions, and deliver definitive interventional therapy. One difficulty associated with current uses of guidewires and catheters for applications in endovascular procedures is ensuring proper positioning and placement of the guidewire and/or catheter during the procedure.

U.S. Pat. Nos. 8,219,180 and 8,725,234 describe systems, devices, and methods for employing endoscopic fiber optic shape tracking sensors. The contents of both U.S. Pat. Nos. 8,219,180 and 8,725,234 are hereby incorporated by reference in their entireties.

### SUMMARY

The following presents a simplified summary in order to provide a basic understanding of some aspects of the innovation. This summary is not an extensive overview of the innovation. It is not intended to identify key/critical elements or to delineate the scope of the innovation. Its sole purpose is to present some concepts of the innovation in a simplified form as a prelude to the more detailed description that is presented later.

According to an aspect of the innovation, a guidewire and/or catheter may be tracked by a fiber optic shape tracking sensor that may incorporate a fiber sensor(s) into the body of a surgical tool (e.g., a vascular guidewire and/or a catheter) during manufacturing of the surgical tool without increasing the size or decreasing the functionality of the surgical tool. This can be exceptionally difficult for very small surgical tools such as vascular guidewires and/or catheters.

In one aspect, the innovation provides devices (e.g., a surgical tool) and methods of making a device that include sensorized guidewires and/or catheters.

In one embodiment, the innovation provides a surgical tool that includes an optical fiber fixedly connected to the surgical tool. In one embodiment, the surgical tool is a sensorized guidewire and/or catheter.

In one embodiment, the innovation provides a method that includes cutting along the shaft of a guidewire or a catheter to create grooves into which a single optical fiber can be fitted. In one embodiment, the single optical fiber has a diameter in the range of about 950 microns to about 200/225 microns. In one embodiment, the single optical fiber has a diameter of 950 microns, 400/425 microns, 300/325 microns, or even 200/225 microns or less. It will be understood that any diameter within the range of about 950 micron to about 200/225 microns falls within the scope of the innovation.

Once fitted into the grooves, the fibers may require a bonding agent or lubricant to allow for slippage during bending of the assembly.

In one embodiment, a plurality of grooves may be created in the outer surface of the surgical tool (e.g., guidewire or catheter). In one embodiment, a total of 3 or 4 grooves can be created in the outer surface of the guidewire or catheter, longitudinally, and spaced 120 degrees (for 3 grooves) or 90 degrees (for 4 grooves) around the surgical tool. Each groove can accommodate one optical fiber sensor. In embodiments with multiple grooves, the availability of multiple optical fibers means that each optical fiber sensor can be a simplified optical fiber sensor. Namely instead of a single optical fiber with 3 quantum dots (QD) spaced at 120 degrees circumferentially around the fiber at each sensorized region the fiber can be now doped with a single quantum dot at each sensorized region.

According to an aspect, the innovation provides sensorized surgical tool and a method of making a sensorized surgical tool, comprising forming a plurality of longitudinal grooves into a shaft of the surgical tool around a circumference of the surgical tool. Each of the grooves is configured to accommodate an optical fiber comprising a fiber optic sensor. One optical fiber is secured into each of the plurality of longitudinal grooves. The surgical tool may be a guidewire or a catheter.

In one embodiment, the plurality of longitudinal grooves is evenly spaced around the circumference of the surgical tool. The forming the plurality of longitudinal grooves may be done by cutting or etching the grooves into the shaft of the surgical tool.

In one embodiment at least three longitudinal grooves are formed in the shaft of the surgical tool around a circumference of the surgical tool. In another embodiment, at least four longitudinal grooves are formed in the shaft of the surgical tool around a circumference of the surgical tool. The grooves may be formed equidistant from each other around the circumference of the surgical tool.

In one embodiment, the fiber optic sensor comprises a fluorophore. In one embodiment, the fluorophore is a quantum dot. The quantum dot may be doped into a cladding of the optical fiber at a sensorized region of the surgical tool.

To accomplish the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

FIG. 1 is an illustration of an example sensorized surgical tool for endovascular navigation in accordance with one or more aspects of the disclosure.

FIG. 2 is an illustration of a portion of example sensorized surgical tool for endovascular navigation in accordance with one or more aspects of the disclosure.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

While specific characteristics are described herein, it is to be understood that the features, functions and benefits of the innovation can employ characteristics that vary from those described herein. These alternatives are to be included within the scope of the innovation and claims appended hereto.

While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

In one or more embodiments a sensorized guidewire and/or catheter for endovascular navigation is provided to facilitate a fiber optic shape tracking sensor for endovascular navigation. The surgical tool (e.g., the guidewire and/or catheter) may also function as a shape tracking sensor that is capable of tracking its own shape, as opposed to a passive tool that relies on fluoroscopy to detect its location and shape inside the vasculature.

According to an aspect, the innovation provides a method of making a sensorized guidewire or a sensorized catheter (or both). In one embodiment, the method may include cutting or etching a groove longitudinally along the shaft of the guidewire or catheter. The groove may accommodate a single optical fiber. In one embodiment, the optical fiber has a relatively small diameter. In one embodiment, the optical fiber has a diameter of 200/225 μm or less. In another embodiment, the optical fiber has a diameter of 300/325 μm or less. In yet another embodiment, the optical fiber has a diameter of 400/425 μm or less.

In one embodiment, the groove may extend substantially the entire length of the shaft of the guide wire or the catheter. In one embodiment the sensorized region of the fiber sensor may cover only a portion of surgical tool. For example, the sensorized region may cover only the distal portion of the surgical tool. This is because the portion of the tool that is external to the vasculature may not need to be sensorized.

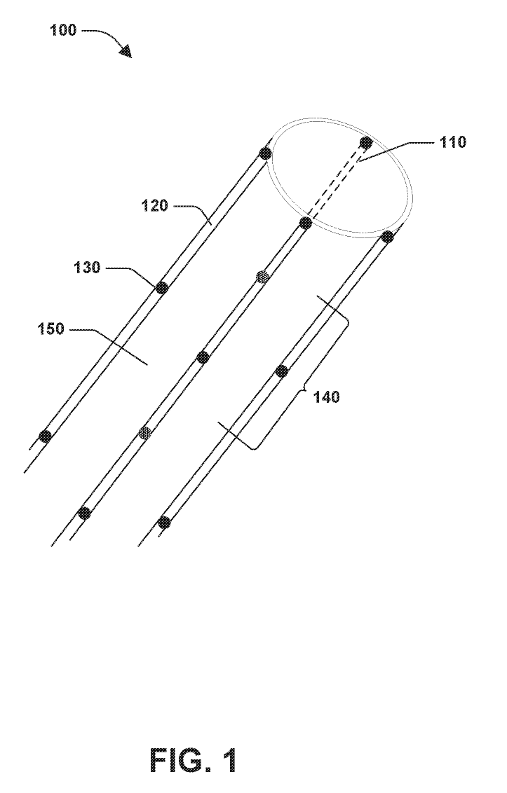

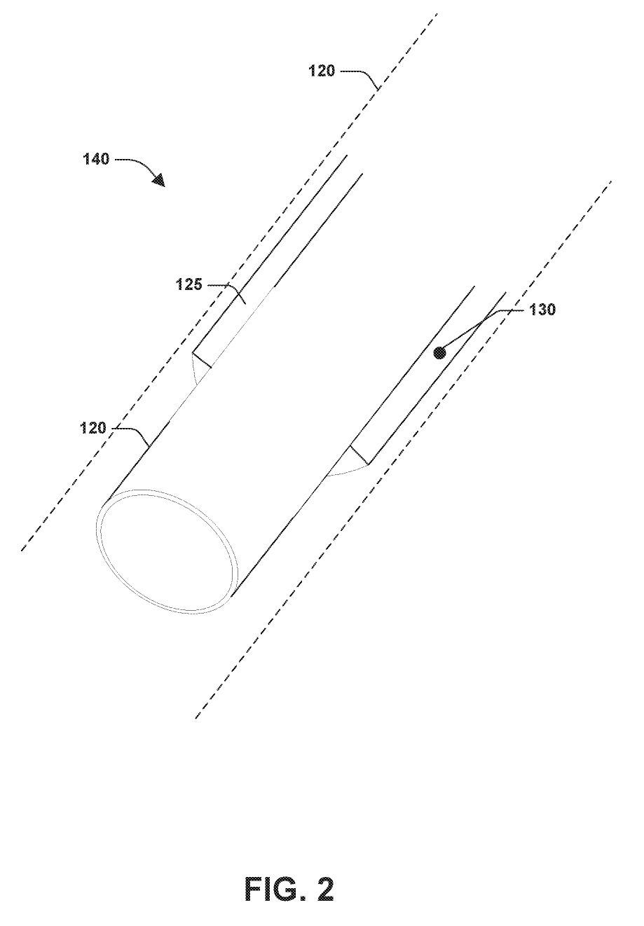

The method may also include securing an optical fiber into the groove. Once fitted into a groove, the optical fiber may require a bonding agent or lubricant to allow for slippage during bending of the surgical tool. In one embodiment, a total of 3 or 4 grooves can be created in the outer surface of the guidewire or catheter, longitudinally and spaced 120 degrees (for 3 grooves) or 90 degrees (for 4 grooves) around the surgical tool (see FIGS. 1 and 2). Each groove can accommodate one optical fiber comprising one fiber sensor. With the availability of multiple fibers around the circumference of the surgical tool, each fiber sensor can now be a relatively simple fiber optic shape tracker sensor.

Present shape trackers include an optical fiber having one or more sets of 3 sensorized zones in the endoscopic fiber optic shape tracker sensors (see U.S. Pat. Nos. 8,219,180 and 8,725,234). This configuration of endoscopic fiber optic shape tracker sensors can be problematic for smaller sensorized surgical tools such as guidewires and catheters because there is no way to adhere the optical fiber to the surgical device without unacceptably increasing the dimensions of the device. The configuration according to the innovation permits the sensorization of the surgical device by creating an area in which the optical fiber may be fixedly attached without significantly increasing the dimensions of the surgical tool. In one embodiment, the groove that is cut or etched into the shaft of the guide wire or the catheter may accommodate a single optical fiber. Because there are multiple grooves and, thus, multiple optical fibers, each optical fiber may include a single fluorophore at each sensorized region. Thus, for example, instead of a single optical fiber with 3 quantum dots spaced at 120 degrees circumferentially around the fiber at each sensorized region, the fiber can be now doped with a single quantum dot at each sensorized region. This would result in one QD emission signal from each activated sensor region, rather than at least three different QD emission signals, thereby allowing a simplified sensor construction, as well as simplified signal analysis. The combined signals from the 3 or 4 fiber sensors in the guidewire or catheter can be analyzed to reconstruct the shape of the surgical tool. As will be understood, the above configuration of grooves is only one possible example of the innovation. Most any configuration of grooves/sensors may be suitable depending on the needs for the surgical tool.

It is to be understood that most any type of fluorophore that permits the detection of light associated with bending in the device may be used to track position and shape according to the innovation. Suitable fluorophores include organic fluorescent dyes. Fluorescent dyes include, but are not limited to, 7-amino-actinomycin D, acridine orange, acridine yellow, auramine O, auramine-rhodamine strain, benzanthrone, 9,10-Bis(phenylethynyl) anthracene, 5,12-Bis(phenylethynyl)naphthacene, CFDA-SE, Calcein, Carboxyfluorescein, 1-Chloro-9,10-bis(phenylethynyl)anthracene, 2-Chloro-9,10-bis(phenylethynyl)anthracene, Coumarin, Cyanine, DAPI, Dark quencher, Dioc6, DyLight Fluor, Ethidium bromide, Fluorescein, Fura-2, Fura-2-acetoxymethyl ester, Green fluorescent protein, Hilyte Fluor, Hoechst stain, Indian yellow, Luciferin, Perylene, Phycobilin, Phycoerythrin, Phycoerythrobilin, Rhodamine, RiboGreen, Rubrene, Ruthenium(II) tris(bathophenanthroline disulfonate), SYBR Green, Stilbene, TSQ, Texas Red, Umbelliferone, and Yellow fluorescent protein.

In some embodiments, the fluorophore is a quantum dot. Quantum dots are nanostructure semiconductor fluorophores with diameters on the order of 2-10 nm. Unlike traditional organic dyes, quantum dots' fluorescence is not reliant on their chemical structure. Quantum dots composed of the same compound can emit fluorescence at different wavelengths by variations in their size.

In one embodiment, the guidewires or catheters can thus be manufactured as a sensorized tool.

According to an aspect, the innovation includes the capability for shape and/or position tracking of the sensorized surgical tool. The sensorized surgical tool may be combined with other medical devices and systems, including imaging hardware and software. In embodiments with multiple sensors, the data from each sensor may be used to provide two- or three-dimensional data for the purpose of visualizing the shape and/or placement of the sensorized surgical tool.

Turning now to FIG. 1, illustrated is an example sensorized surgical tool 100 in accordance with an embodiment disclosed herein. The surgical tool 100 has grooves 110 cut or etched into the shaft 150 of the surgical tool An optical fiber shape sensor comprising an optical fiber 120 and fluorophore 130 may be embedded in a groove 110. In one embodiment, the fluorophore 130 may be in the cladding of the optical fiber as show in FIG. 2. In one embodiment, the fluorophore may be a quantum dot.

Turning now to FIG. 2, a portion 140 of FIG. 1 is illustrated as an example of a sensorized surgical tool in accordance with an embodiment disclosed herein. The optical fiber 120 comprises a cladding 125. A fluorophore 130 may be included within the cladding 125. The optical fiber 120 is secured within a groove 110 that is formed in the shaft of the surgical tool. The optical fiber may be secured by most any means including by an adhesive, a deformable member (e.g., a rubber or plastic wedge, gasket, etc.), by friction fit, or any reasonable mechanical means.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

1. A method of making a sensorized surgical tool, comprising:
forming a plurality of longitudinal grooves into a shaft of the surgical tool around a circumference of the surgical tool, wherein the groove is configured to accommodate an optical fiber comprising a fiber optic sensor, the sensor comprising a fluorophore, wherein the fluorophore is within a cladding of the optical fiber at a sensorized region of the surgical tool; and
securing one optical fiber into at least one of the plurality of longitudinal grooves.

2. The method of claim 1, wherein the plurality of longitudinal grooves are evenly spaced around the circumference of the surgical tool.

3. The method of claim 1, wherein the fluorophore comprises a quantum dot doped into the cladding of the optical fiber.

4. The method of claim 1, wherein the surgical tool is a guidewire.

5. The method of claim 1, wherein the surgical tool is a catheter.

6. The method of claim 1, wherein forming the plurality of longitudinal grooves is done by cutting the grooves into the shaft of the surgical tool.

7. The method of claim 1, wherein forming the plurality of longitudinal grooves is done by etching the grooves into the shaft of the surgical tool.

8. The method of claim 1, wherein at least three longitudinal grooves are formed the shaft of the surgical tool around a circumference of the surgical tool.

9. The method of claim 1, wherein at least four longitudinal grooves are formed in the shaft of the surgical tool around a circumference of the surgical tool.

10. A sensorized surgical tool, comprising:
a plurality of longitudinal grooves formed into the surgical tool around a circumference of the surgical tool;
a plurality of optical fibers, and
at least one fiber optic sensor comprising a quantum dot, wherein the quantum dot is doped into a cladding of at least one of the plurality of optical fibers,
wherein each longitudinal groove of the plurality of longitudinal grooves accommodates one optical fiber of the plurality of optical fibers.

11. The sensorized surgical tool of claim 10, wherein the surgical tool is a guidewire.

12. The sensorized surgical tool of claim 10, wherein the surgical tool is a catheter.

13. The sensorized surgical tool of claim 10, wherein at least one of the fiber optic sensors comprises a fluorophore other than a quantum dot.

14. The sensorized surgical tool of claim 13, wherein the fluorophore comprises an organic fluorescent dye.

15. The sensorized surgical tool of claim 14, wherein the fluorophore is doped into the cladding of the optical fiber at a sensorized region of the surgical tool.

16. The sensorized surgical tool of claim 10, wherein at least three longitudinal grooves are formed in the shaft of the surgical tool around a circumference of the surgical tool.

17. The sensorized surgical tool of claim 10, wherein at least four longitudinal grooves are formed in the shaft of the surgical tool around a circumference of the surgical tool.
